(19) United States
(12) Patent Application Publication
(10) Pub. No.: US 2026/0309349 A1
(43) Pub. Date: Oct. 8, 2026

(54) Devices and Methods for Multi-Layer Composite Laminar Flow

(71) Applicant: Embrient, Inc, San Diego, CA (US)

(72) Inventor: Kyong-su Son, San Diego, CA (US)

(73) Assignee: Embrient, Inc, San Diego, CA (US)

(21) Appl. No.: 19/638,056

(22) Filed: Apr. 2, 2026

Related U.S. Application Data
(60) Provisional application No. 63/783,205, filed on Apr. 3, 2025.

(51) Int. Cl. F24F 9/00; C12M 1/00

(52) U.S. Cl. CPC F24F 9/00; C12M 1/005

(57) ABSTRACT

An incubator includes a flow conditioner that produces a composite air veil in which adjacent sections and outer sections relative to static ambient air have a flow velocity difference that is below a shear force velocity difference. Advantageously, such air veils have improved stability and can even be operated at velocities that would otherwise lead to significant turbulence.

This application claims the benefit of our US Provisional Patent Application with the serial number 63/783,205, which was filed Apr. 3, 2025, and which is incorporated by reference herein.

### FIELD OF THE INVENTION

The field of the invention is devices and methods for providing a multi-layer composite air veil, especially as it relates to use of such air veils in incubators.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Air curtains are used widely to create air barriers that reduce leakage of air between a climate-controlled room and its surrounding environment. Air curtains also help reduce insects or dust to enter the climate-controlled room. For example, air curtains are often implemented in the doorways of retail and commercial spaces to maintain the internal atmosphere in the space and reduce the ingress of insects. Air curtains are also found in various industrial space heating and cooling systems to help reduce unnecessary energy loss.

In more recent applications, the use of air curtains with laminar or non-turbulent flow has been implemented in incubators, providing air veils over a door opening of the incubators to advantageously protect the internal atmosphere from mixing with the ambient atmosphere. See, for example, US Patent No. 10,900,010, which is incorporated by reference herein. Such use may be particularly advantageous where the internal atmosphere of an incubator must be carefully controlled to establish and maintain precise experimental conditions.

However, most known laminar flow devices suffer from several drawbacks. For example, “because of the high airflow velocity, columnar air curtains have several shortcomings, such as the nonuniform distribution of air, deviation from the axis, and the lack of a basis for determining the volume of the air curtain unit. Existing research cannot systematically explain the factors that directly influence these shortcomings” (see e.g., Xiaochuan Li et al., "Air curtain dust-collecting technology: Influence factors for air curtain performance," Journal of Wind Engineering and Industrial Aerodynamics, Vol. 218, 2021, Article No. 104780, ISSN: 0167-6105). Proposed solutions have included an air curtain system that used pipeline air curtains with a nozzle diameter of 1 mm and a nozzle distance of 15 mm which exhibited a relatively good barrier effect and barrier efficiency of 55–60%. Id. As will be readily appreciated, such barrier efficiency is not optimal. In addition, and particularly where two laminar or non-turbulent flow air curtains are operated in parallel, interference between the curtains may introduce turbulence or vortices that may then compromise the barrier function. Still further, and especially where air velocity of an air curtain is relatively high, turbulence or vortices at the air flow boundary to static air is almost unavoidable.

Thus, even though various devices and methods of using air curtains are known in the art, all or almost all of them suffer from several drawbacks. Therefore, there remains a need for improved devices and methods of providing air curtains (preferably with laminar or non-turbulent air flow) that can be effectively operated at relatively high air velocities.

### SUMMARY OF THE INVENTION

The inventive subject matter is directed to various devices and methods in which a composite air veil is used in an incubator. Advantageously, such composite air veil or air veils will effectively shield and fluidly isolate the contents of the internal container of the incubator even at high air speed without issues otherwise encountered arising from turbulence or vortices at boundary layers to the surrounding atmosphere.

In one aspect of the inventive subject matter, the inventors contemplate an incubator that has a housing that encloses an internal container, wherein the housing and the internal container have an opening that allows access to the inside of the internal container. A flow conditioner is coupled to the housing and/or internal container and configured to form a composite air veil across the opening, wherein the composite air veil has a thickness and a velocity gradient across the thickness, and wherein the flow velocity difference between two adjacent sections within the thickness is below a shear force velocity difference.

In some embodiments, the flow conditioner includes a flow control element that receives an air flow and induces a centrifugal or deflecting force in the air flow in an amount sufficient to produce a density gradient in the air flow to so produce a continuous velocity gradient across the thickness of the composite air veil. In other embodiments, the flow conditioner includes a flow control element that receives an air flow and induces pressure gradient in the air flow to thereby produce the velocity gradient across the thickness of the composite air veil (e.g., the velocity gradient may be a continuous velocity gradient or a stepped velocity gradient.

In further contemplated embodiments, the flow conditioner may be configured to form two composite air veils across the opening. In such case, two composite air veils will typically flow along two substantial parallel hypothetical planes. As will be appreciated, the first of the two composite air veils may have an ascending velocity gradient in the direction from the inside to the outside of the internal container, and the second of the two composite air veils may have a descending velocity gradient in the direction from the inside to the outside of the internal container. Where desired, the flow conditioner may be configured to allow independent operation of the first and the second composite air veils.

Most typically, but not necessarily, the incubator will include a movable door covering the opening, and the door will cover the composite air veil and internal container. It is still further contemplated that the thickness of the composite air veil is between 1 cm and 10 cm, and/or that the shear force velocity difference will be at least 0.4 m/s. Where desired, the flow conditioner may be configured to allow some of the lower velocity air in the composite air veil to move into the internal container. For example, such air feed may be achieved by a movable element or flow restrictor that forces the portion of lower velocity air in the composite air veil into the internal container.

In yet further embodiments, the incubator includes a recirculation conduit that is configured to recycle at least some of the air in the composite air veil. For example, such the recirculation conduit may comprise tubing with a width or diameter that is less than the width of the opening of the internal container. As will be readily appreciated, the incubators presented herein will typically be a controlled atmosphere incubator. As such, it is contemplated that the composite air veil may have a composition other than ambient air. Where desired, the incubator may include a control system that is configured to adjust a flow rate of air in the composite air veil, and/or that is configured to maintain an atmospheric parameter (e.g., oxygen concentration, humidity, temperature, etc.) in the internal container.

Therefore, in another aspect of the inventive subject matter, the inventors also contemplate a flow conditioner for forming a composite air veil that includes a flow control grate coupled to and in fluid communication with a multi-channel unit having a plurality of flow channels. Most typically, the flow control grate comprises a plurality of pass-through elements that are arranged in rows and columns, wherein pass-through elements in a first row have a lower flow resistance to air flowing through the flow control grate than pass-through elements in a second row adjacent to the first row.

In some embodiments, each of the pass-through elements has the same unit size, and wherein each of the pass-through elements in the first row have a larger pass-through area per unit size as compared to a pass-through area per unit size of the pass-through elements in the second row. Among other options, the pass-through area may be formed from one or more openings, for example, with each of the openings within a pass-through area having equidistant spacing. Additionally, it is contemplated that each column of the pass-through elements may have an increasingly lower pass-through area per unit size at a more peripheral location. In further embodiments, each column of the pass-through elements has an increasing pass-through area per unit size. It is generally contemplated (but not necessary) that each pass-through element is fluidly coupled to a single flow channel. Preferably, at least some of the flow channels may comprise a dead-space filling.

Alternatively, in another aspect of the inventive subject matter, the inventors also contemplate a flow conditioner for forming a composite air veil that comprises a flow control diverter coupled to and in fluid communication with a multi-channel unit having a plurality of flow channels. Typically, the flow control diverter will include an angled or rounded element that imparts a centrifugal or deflecting motion to an air flow, wherein the centrifugal or deflected air flow produces a density gradient in the air flow such that adjacent flow channels receive progressively increasing volumes of air.

For example, the flow control diverter may include an angled element or a rounded element, and/or it is contemplated that the flow control diverter may be configured to direct air such that inflowing air and exiting air move at an angle of about 90 degrees.

Consequently, the inventors also contemplate a method of maintaining atmospheric conditions within an incubator having a housing that encloses an internal container, wherein the housing and the internal container have a door covering an opening that allows access to an inside of the internal container. In such method, a flow conditioner is operated when the door is in an open position, wherein the flow conditioner forms a composite air veil moving across the opening. As noted before, the composite air veil has a thickness and a velocity gradient across the thickness, and the flow velocity difference between two adjacent sections within the thickness is below a shear force velocity difference.

In some embodiments, the flow conditioner produces a composite air veil that has a symmetrical velocity gradient. In further embodiments, contemplated incubators may also include a second flow conditioner that forms a second composite air veil moving substantially parallel to the composite air veil moving across the opening. As will be appreciated, the flow conditioner may include a flow control element that receives an air flow and induces a centrifugal or deflecting force in the air flow sufficient to produce a density gradient in the air flow to thereby produce a continuous velocity gradient across the thickness of the composite air veil. Alternatively, the flow conditioner may include a flow control element that receives an air flow and induces a pressure gradient in the air flow to thereby produce the velocity gradient across the thickness of the composite air veil. Most typically, a control system will be used to control air flow in the composite air veil, and/or to control composition of air in the composite air veil. Advantageously, the flow velocity in the fastest sections of the composite air veil may be well above 2 m/s.

Various objects, features, aspects, and advantages of the inventive subject matter will become more apparent from the following detailed description of preferred embodiments, along with the accompanying drawing figures in which like numerals represent like components.

### Brief Description of The Drawing

FIG. 1 is an exemplary schematic illustration of a controlled-atmosphere incubator with a flow conditioner that produces a composite air veil.

FIG. 2 is an exemplary schematic illustration of the effect of a flow conditioner using discontinuous air density to produce a continuous flow velocity gradient.

FIG. 3 is an exemplary schematic illustration of the effect of a flow conditioner using a pressure gradient to produce a stepped flow velocity gradient.

FIG. 4 is an exemplary schematic illustration of flow resistance for flow conditioners operating with a flow control grate, and exemplary flow control grates to produce pressure gradients.

FIG. 5 is a photograph of an exemplary flow conditioner with a flow control grate partially removed from a muti-channel unit.

FIG. 6 is an exemplary schematic illustration of a flow channel with dead-space filling. As depicted, gas flow enters the flow channels and is not vacuumed into the corners or outer walls of the channel (the dead-space) because of the dead-space filling.

FIG. 7 is an exemplary schematic illustration of a flow channel with serrated edges positioned along at the exit side of the flow channel, reducing potential turbulence.

### DETAILED DESCRIPTION

The inventors have now discovered that various problems ordinarily encountered with conventional air veils, and especially turbulence at the boundary layer between the air veil and static ambient air, can be readily overcome by generating a composite air veil, even where such air veils operate at relatively high speeds. Advantageously, such composite air veils have an air flow velocity gradient across the thickness of the air veil such that a flow velocity difference between two adjacent sections within the thickness and between the outermost section and static ambient air is below a shear force velocity difference, which is typically about 0.4 m/s.

In this context it should be appreciated that all or almost all of the currently known air veil systems face challenges with turbulence when operating at relatively high speeds or when multiple air veils are used side by side. Consequently, such systems often encounter disruption of the intended barrier function and may have reduced effectiveness in maintaining temperature control or in preventing contamination. This is primarily caused by the interaction between the high-velocity airflow and adjacent stationary air. Furthermore, when multiple air veils operate in adjacent positions, their airflows often interfere with one another, creating unpredictable turbulence patterns that once more diminish their performance and can lead to inefficiencies or gaps in the protective barrier.

Viewed from a different perspective, the inventors contemplated devices, systems, and methods for flow conditioners that are able to produce multiple adjacent layers of laminar or non-turbulent flow, wherein the difference in velocity between any two adjacent layers does not exceed a predetermined threshold. Likewise, the inventors also contemplate that the difference in velocity between the outermost layers of the laminar or non-turbulent flow and adjacent stationary air will also not exceed the predetermined threshold. It is contemplated that, so long as the outermost layers of flow do not interact turbulently with stationary air, and adjacent inner layers do not interact turbulently with one another, the velocity of the inner layers can be increasingly higher towards the midline of the layers virtually without any turbulence. After numerous experiments, the inventors discovered that the composite air veils provided non-turbulent or laminar flow with significantly higher barrier strength, stability, and maximum velocity, while significantly reducing or even entirely avoiding turbulence as compared to simple air veils known in the art.

Barrier strength in the context of laminar flow typically refers to the ability of a laminar flow to resist disturbances or penetration that could disrupt the smooth layers of air movement. It may further describe how well the flow maintains its non-turbulent or laminar characteristics against external forces or obstacles. Barrier strength may be measured using several methods. For example, a calculation of the Reynolds number may be used to predict whether an air flow or gas flow will remain laminar to transition to turbulence. Such calculation, along with the knowledge of the specific composition and condition of the air such as temperature, humidity, velocity, gas composition, etc., may be used in computational fluid dynamics models for air flow. Alternatively, or additionally, visible smoke may be introduced into the non-turbulent or laminar flow to allow observation of transitions from laminarity to turbulence. The smoke may be illuminated using a bright light source to enhance visibility. This method and/or other methods may be used in conjunction with a high-speed camera combined with image processing software to record the motion of smoke in real time. In yet other examples, an observer can also measure fluctuations in velocity or pressure over time to calculate the intensity of turbulence. Regardless of the manner of data acquisition, contemplated composite air veils are particularly suitable for isolation and protection of an otherwise closed environment where the closed environment has an opening that allows for access or ingress, and where the environment is atmospherically controlled (e.g., to maintain specific oxygen centration, temperature, humidity, etc.)

Therefore, in one embodiment, the inventors especially contemplate an incubator that has a housing that encloses an internal container, wherein the housing and the internal container have an opening that allows access to the inside of the internal container. A flow conditioner is coupled to the housing and/or internal container and configured to form a composite air veil across the opening, wherein the composite air veil has a thickness and a velocity gradient across the thickness, and wherein the flow velocity difference between two adjacent sections within the thickness is below a shear force velocity difference. For example, FIG. 1 schematically shows incubator 100 in which a housing 110 encloses an inner container 120 with opening 122. Space 130A and 130B for separate air recirculation comprise respective fans (not shown) that are fluidly coupled to flow conditioner 140 and flow receiver 140’ to enable a recirculating air flow in the respective spaces. In this example, the flow conditioner 140 produces two adjacent composite (multi-layer) air veils 142. Here, first of the two composite air veils has an ascending velocity gradient in the direction from the inside to the outside of the internal container, and the second of the two composite air veils has a descending velocity gradient in the direction from the inside to the outside of the internal container. Where it is desired to direct at least some of the air flow into the inner container, a movable element 144A (e.g., vane, jet, etc.) or flow restrictor 144B may be used. Door 160 is preferably positioned and coupled to the incubator to close in a sliding or composite motion (horizontally and vertically away from the opening) such as not to introduce vertices otherwise encountered with doors that rotate/pivot about a peripheral axis. As will be readily appreciated, a controller 150 will be operationally coupled to the incubator to control flow velocity of the composite air veil, movement of the door, as well as other operational parameters such as temperature, humidity, gas composition, etc. Moreover, it should be recognized that while FIG. 1 depicts the two recirculation spaces for the two respective air veils as continuous spaces between wall elements, it should be noted that at least part of one or both recirculation spaces can be formed as tubing, round or rectangular smaller conduits that will then terminate in a distribution element upstream of the respective flow control elements and/or that may connect downstream of the respective flow receivers. While not limiting to the inventive subject matter, it is further contemplated that the incubators presented herein will not only comprise one or more (radial) fans or propellers but that the air from returning to the recirculation space may also be assisted by one or more suction fans.

In some embodiments, exemplary incubators suitable for use herein are disclosed in US Patent Nos. US 10,900,010, US 11,072,773, US 11,203,737, US 11,365,385, US 11,634,681 B2, Patent Publication No. US 2023/0242,861 A1, US 11,884,910 B2, and PCT No. WO 2021/046185, all of which are incorporated by reference herein. Additionally, the inventors further contemplate that the devices and methods presented herein may be suitable for many other applications, including but not limited to commercial spaces, retail store entrances, shopping malls, supermarkets and convenience stores, warehouses and storage facilities, cold storage rooms, meat/produce freezers, office building entrances, restaurants and cafes, refrigerators, kitchens, factories, hospital entrances, sterile environments (research labs, surgery rooms, etc.), pharmacies, schools and university buildings, event centers, gyms, animal shelters or livestock facilities, greenhouses and other farm-related applications, homes, indoor swimming areas, or any other suitable applications.

The flow rates may also be dynamically adjusted during operation. For example, when an incubator door is opened, the flow rates may be temporarily increased to strengthen the air curtain barrier. Once the door is closed, the flow rates may be reduced back to lower levels. The control system may employ PID control algorithms to maintain stable flow conditions. Sensors like thermal mass flow meters, oxygen sensors, and humidity sensors may provide feedback. Actuators like proportional solenoid valves may modulate individual channel flow rates.

Referring back to flow conditioners contemplated herein, it is typically preferred (but not needed) that the flow conditioner includes a flow control element that is configured to receive an air flow and to induce a centrifugal or deflecting force in the air flow sufficient to produce a density gradient in the air flow to thereby produce a continuous velocity gradient across the thickness of the composite air veil. Alternatively, contemplated flow conditioners may also include a flow control element that is configured to receive an air flow and to induce a pressure gradient in the air flow to thereby produce a velocity gradient across the thickness of the composite air veil. As is discussed in more detail below, the velocity gradient may be a continuous velocity gradient or a stepped velocity gradient. It should further be appreciated that a single flow conditioner may produce a single composite air veil with a linear ascending or a linear descending gradient or a composite air veil with a symmetric gradient (where the gradient is ascending and then descending as also shown in the detail below).

Thus, it should be recognized that two separate flow conditioners can also produce two separate and adjacent composite air veils (e.g., having linear ascending or descending gradient or with symmetric gradient). Where two composite air veils are used, it is generally preferred that the distance between the air veils as measured center-to-center between the air veils is between 5 mm and 1.5 cm, or between 1.5 cm and 5 cm, or between 5 cm and 10 cm, or between 10 cm and 25 cm, and even higher. less than 10 cm, less than 5 cm…). Therefore, the distance of two air veils may be at least 5 mm, or at least 1.5 cm, or at least 5 cm, or at least 7.5 cm, or at least 10 cm, or at least 15 cm, or at least 20cm, but typically less than 50 cm, or less than 30 cm.

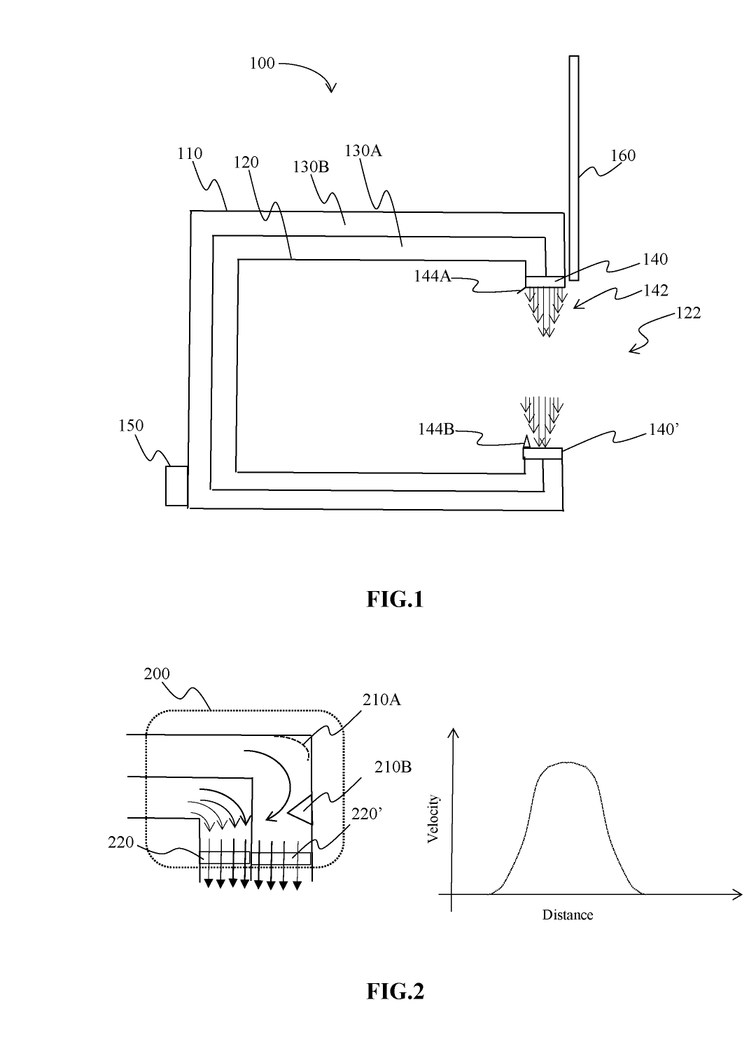

FIG. 2 depicts a schematic illustration of an exemplary flow conditioner in which a density gradient (via centrifugal or deflective motion) is produced. Here, the flow control diverter 200 is fluidly coupled to and in fluid communication with a multi-channel unit 220 and 220’ having a plurality of flow channels. In some embodiments, the flow control diverter comprises an angled or rounded element that is configured to impart a centrifugal or deflecting motion to an air flow such that the centrifugal or deflecting air flow produces a density gradient in the air flow and such that adjacent flow channels receive progressively increasing volumes of air. As will be readily appreciated, the flow control diverted in such devices may have a variety of shapes, but particularly preferred shapes comprise an angled element or rounded element.

With further reference to FIG. 2, flow conditioning element 200 is formed in part from recirculation spaces and at least one of the recirculation spaces includes a rounded element 210A and/or an angled element 210B. In this context, it should be appreciated that air behaves as a fluid and that deflecting or centrifugal forces will induce a density gradient in which air flowing in the ‘outside lane’ of a turn has a higher density that air flowing in the ‘inside lane’ as is schematically shown in FIG. 2. The redirected air is then fed into multi-channel unit 220 and 220’ having a plurality of flow channels to so form a composite air veil. The right panel of FIG. 2 depicts an air velocity profile across the thickness of the composite air veil where no adjacent sections exceed shear force velocity difference. In most embodiments, the flow control diverter is configured to direct air such that inflowing air and exiting air move at an angle of about 90 degrees relative to each other.

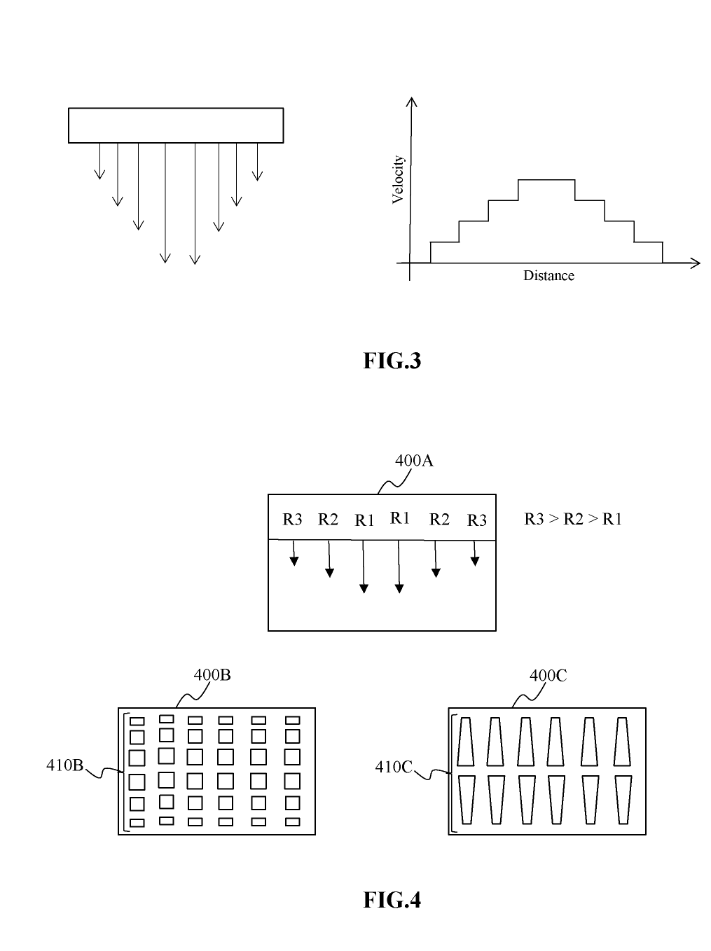

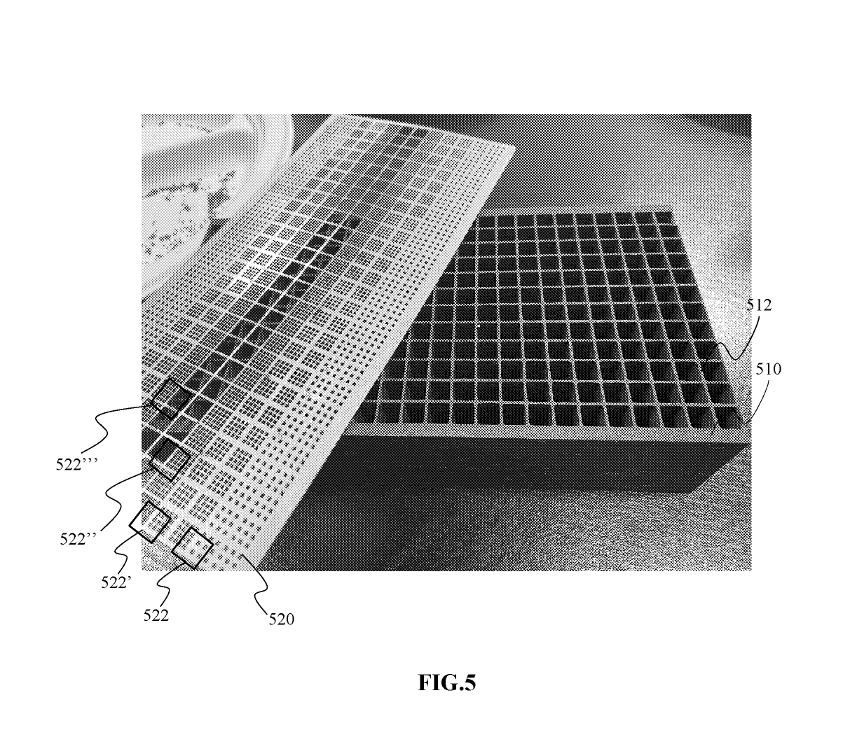

On the other hand, where the flow conditioner employs a pressure gradient, the flow conditioner produces a stepped air velocity profile across the thickness of the composite air veil as is exemplarily shown in the right panel of FIG. 3. The left panel depict an exemplary air flow output where shorter arrows denote slower flow rates. As can be seen from this schematic illustration, the composite air veil has an ascending air flow gradient and a descending air flow gradient. Once more, the flow differences between adjacent sections will not exceed shear force velocity difference. As is schematically shown in FIG. 4, a pressure differential can be achieved in either a stepped manner using a grate having widening slots as is schematically shown in left panel, producing different sections of flow resistance as noted in the upper panel (with higher resistance on the outside sections and lower resistance towards a hypothetical midline. Where a continuous pressure gradient is desired, slots with increasing width can be used as shown in right panel of FIG. 4. As noted above, it should be appreciated that the flow conditioner with pressure gradients can be configured as a single unit or as two separate units.

For example, FIG. 5 depicts an exemplary and partially disassembled flow conditioner. Here, the flow conditioner is partly disassembled to show flow control grate 520 that once assembled will be coupled to and will be in fluid communication with a multi-channel unit 510 having a plurality of flow channels 512. In this example, the flow control grate 520 comprises a plurality of pass-through elements 522, 522’, 522’’, and 522’’’ that are arranged in rows and columns. As can be seen in FIG. 5, the pass-through elements 522’’’ in the first row (as counted from a hypothetical midline) produce a lower flow resistance to air flowing through the flow control grate 520 than pass-through elements 522’’ in a second row adjacent to the first row. The pass-through elements 522’ in the third row have yet a higher flow resistance than those in the second row, and pass-through elements 522 in the fourth (outer) row have the highest flow resistance. As will be readily appreciated, such flow conditioner will produce a stepped flow resistance and stepped air velocity gradient.

In the example of FIG. 5, each of the pass-through elements has the same unit size, and each of the pass-through elements in one row have a larger pass-through area per unit size as compared to a pass-through area per unit size of the pass-through elements in an adjacent row. As will be readily appreciated, the pass-through area can include one or more openings that may be square shaped, rounded, or configured as micro-jets extending into the channels of the multi-channel unit. Moreover, it is contemplated that such openings may be spaced equidistantly or follow a predetermined non-equidistant pattern. Most typically, each column of the pass-through elements may have an increasingly lower pass-through area per unit size at a more peripheral location. Alternatively, or additionally, each column of the pass-through elements may have an increasing pass-through area per unit size. Regardless of the openings and geometry of the pas-through elements, it is generally preferred (but not necessary) that each pass-through element will provide to a single corresponding flow channel.

Most typically, contemplated flow conditioners (operating with a pressure or density gradient) will include a multi-channel unit with a plurality of flow channels. In such devices, a row of flow channels will therefore produce an individual air veil within the composite air veil, and adjacent rows will produce adjacent air veils having a velocity difference that is below a shear force velocity difference. Of course, it should be recognized that the flow channels need not have a square shape as exemplarily depicted in FIG. 5 but may have any geometry suitable to produce a single air veil within the composite air veil. Moreover, it is generally contemplated that the air exiting the flow channel will be at least non-turbulent, or more preferably exhibit laminar flow. Among other options, contemplated flow channels can have a width ranging from 5-20 mm, with 10-15 mm being preferable. Their height may be between 20-100 mm, preferably 40-60 mm, while the length can range from 50-200 mm, with 100-150 mm being optimal. The cross-sectional shape of the channels may be square, rectangular, or rounded rectangular. There can be anywhere from 10-100 channels, with 20-50 being preferable, spaced 1-5 mm apart.

For the grate openings, they may be circular, square, or hexagonal in shape. The diameter or width of these openings can range from 1-10 mm, with 2-5 mm being preferable. The number of openings per pass-through element varies, with midline elements having 1 opening, while elements further from the midline increase from 2 to 10 openings. The spacing between openings is typically 0.5-2 mm. The total open area percentage ranges from 30-70%, increasing from outer to inner elements. The grate itself may be 1-5 mm thick and constructed from perforated metal or plastic. The overall dimensions of the flow conditioning unit and grate can range from 100 mm to 100 cm wide by 50-200 mm tall. Of course, it should be recognized that these specific dimensions can be optimized based on the desired flow characteristics and application requirements. Generally, smaller openings and channels provide more precise flow control, while larger dimensions allow for higher overall flow rates.

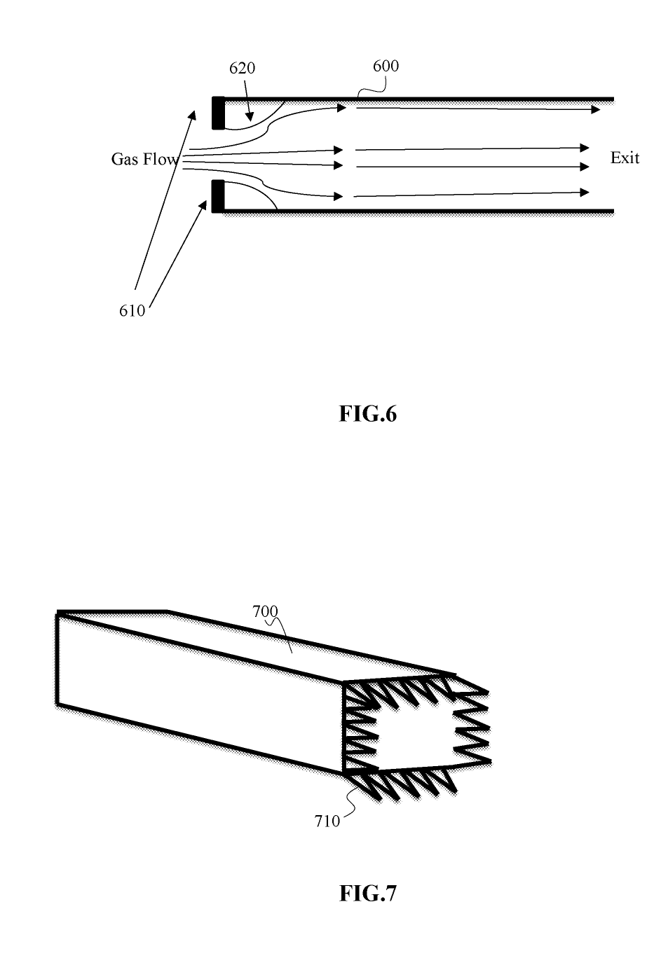

As will be readily appreciated, the flow conditioner may be manufactured using various methods and materials. For example, a flow conditioning unit housing may be formed of molded plastic, sheet metal, or other rigid materials. The flow channels may be created by inserting baffles or dividers into the housing. The grate may be made of perforated metal, plastic, or other materials with precisely sized and positioned openings. Assembly may involve bonding or mechanically fastening the components together.

Where desired, at least some of the flow channels may further comprise a dead-space filling as exemplarily shown in FIG. 6. Here, flow channel 600 is partially covered by a pass-through element 610 permitting air flow (from left to right in this figure) through the opening in the pass-through element 610. Dead-space filling 620 is placed an upstream portion of the flow channel to thereby discourage formation of flow vortices at the transition and to encourage non-turbulent or laminar flow. Optionally, and as shown in FIG. 7, an exemplary downstream end of a flow channel 700 has undulating or spike-shaped termini to maintain non-turbulent or laminar flow.

Therefore, it should be appreciated that flow conditioners can be manufactured that produce a composite air veil with a number of adjacent individual air veils having different flow velocities. Most preferably, the velocity difference between two adjacent individual air veils will be at, near, and more typically below the shear force velocity difference. Typically, and considering air with a nitrogen content of between 70 vol% and 99.99 vol%, and oxygen content of about 24 vol% to 0.01 vol%, and a humidity of between about 15-80% (relative humidity), and a temperature of about 15 to 50 °C, the shear force velocity difference will be about 0.4 m/s for a non-turbulent or laminar flow. Therefore, contemplated shear force velocity differences between individual air veils in the composite air veil and the outermost ail veil of the composite air veil and static ambient air will be between about 0.4-0.35 m/s, or between 0.35-0.25 m/s, or between 0.25-0.15 m/s, and even less. Viewed form a different perspective, it is contemplated that the shear force velocity differences will be below a difference that induces turbulent flow.

In contemplated embodiments, and depending on the specific uses and requirements, a composite air veil will include between 3 and 15 individual air veils, such as between 3 and 5, or between 5 and 7, or between 7 and 9, or between 9 and 11. Therefore, and with regard to minimum and maximum velocities of composite air veils, it is contemplated that the outermost air veil facing static ambient air will have a flow velocity of equal or less than 0.4 m/s, equal or less than 0.35 m/s, equal or less than 0.3 m/s, equal or less than 0.25 m/s, or equal or less than 0.2 m/s, but higher than 0.5 m/s, or 1.0 m/s, or 1.5 m/s. Conversely, the innermost air veil(s) in the composite air veil may have a flow velocity of at least 1.2 m/s, or at least 1.5 m/s, or at least 1.8 m/s, or at least 2.1 m/s, or at least 2.5 m/s, or at least 3 m/s, and even higher. In this context, it should be appreciated that such high velocities (e.g., >2.0 m/s) in single layer air veils would normally lead to significant turbulence with static outside air. Viewed from a different perspective, contemplated composite air veils will provide significant barrier function even at high velocities. In still other embodiments, the difference in velocity between airflows may also be zero. It will be readily understood that the maximum difference in velocity between air flows may be calculated for individual flow conditioners depending on various factors, such as the distance traveled by the air veil, the density and/or the content of the gas, the pressure of the ambient environment, and/or other factors that may affect the stability and/or barrier effect of a laminar flow veil. Viewed from a different perspective, the inventors contemplate that flow conditioners and/or methods disclosed herein may provide a flow of air with a Reynolds number less than 900, or less than 1000, or less than 1200, or less than 1400, or less than 1500, or less than 1600, or less than 1700, or less than 1800, or less than 1900, or less than 2000.

It should further be appreciated that at least a portion of air flowing through the flow conditioner may be directed into the inner container of the incubator. For example, a plurality of pass-through elements on the outer boundaries of the composite flow conditioner may be configured to direct gas flow into an internal container of an incubator using a movable element and/or flow restrictor as described above. Alternatively, at least a portion of the flow conditioner may be moved/positioned to direct some of the circulating air into the container. In addition to providing an air curtain to protect and maintain the internal environment of the incubator, this may allow the direction of certain gases into the incubator to adjust or maintain desirable atmospheric conditions without requiring a separate conduit for delivering gas to the internal container. Alternatively, or additionally, the composite flow conditioner may work in conjunction with a separate conduit to deliver gas to a designated space such as an internal container of an incubator. In another aspect, the composite flow conditioner may be configured to both deliver gas to the incubator and to circulate air in the internal container as needed. For instance, if the gas needs to mix with other substances in the incubator, such as biological systems, chemical reactions, or the general atmosphere within the incubator, the composite flow conditioner may be configured to blow the gas into the incubator at higher (e.g., turbulent) speeds or pressures to promote mixing.

In further embodiments, and as already noted above, the inventors contemplate that the incubators as described herein may be coupled to a control unit. For example, a control unit may be configured to allow a user to selectively configure various parameters of the air composition and/or air flow in the composite air veil. In one embodiment, the control unit may allow a user to specify that the maximum velocity of the laminar flow is 1.8 m/s. In another embodiment, the control unit may allow a user to configure flow limiters in different flow channels. The control unit may be coupled to the flow conditioner to configure flow parameters. This unit may include a microprocessor, memory, and I/O interfaces. Flow sensors may be integrated to provide feedback on gas velocities in each channel. Proportional valves or other flow control mechanisms may be actuated by the control unit to adjust individual channel flow rates.

The inventors further contemplate that at least a portion of the flow conditioner may be configured to receive air from an external environment and/or ambient air, while another portion may be configured to receive a pre-mixed gas to control atmospheric conditions inside the inner container. Therefore, the composite flow conditioner may be configured to form a primary and a secondary air veil that are substantially parallel and extend across an opening. Where desired, for contemplated systems and methods herein, a primary air veil and/or the secondary air veil is a directional veil or a laminar flow veil. Preferably, the primary air flow control device further comprises a movable vane or other element that controls the direction of the primary air veil.

Devices for air veils and/or parallel air curtains in the prior art lack several advantageous features seen in the invention disclosed herein. For example, in Patent Publication No. DE 29818548 U1, the disclosure makes no mention of any differences in air flow velocity between the individual air veils or channels. Indeed, the patent states that “the air curtain outlet nozzle is divided into a plurality of identical, vertically running channels,” and the exit speed of the air curtain from the entire air curtain outlet nozzle is specified as 3 to 15 m/s, with 3 to 4 m/s being preferred for smaller rooms. Therefore, the prior art, as represented by this patent, describes air flow curtains as being uniform across the entire width of the air curtain outlet nozzle.

In still further contemplated embodiments, the flow conditioner may be coupled to gas supply and mixing systems of the incubator or other apparatus. These systems may provide pre-mixed gas compositions to be flowed through the conditioner. For example, a nitrogen-rich gas stream may be supplied to inner flow channels while an oxygen-rich stream is supplied to outer channels. This allows creation of graduated gas composition profiles across the air veil. Control systems may be integrated to regulate gas flow through the device. Sensors may monitor parameters like gas composition, humidity, temperature, and flow rates at various points. A controller may use this data to adjust flow rates through individual channels via flow limiters or valves. This enables dynamic tuning of the air veil properties.

For example, specific gas mixtures that may be used include: 5% CO2, 95% air for cell culture applications; 5% O2, 5% CO2, 90% N2 for hypoxic conditions; 100% N2 for anaerobic environments; up to 100% O2 for hyperoxic conditions; or any other combination of any gases. The gas mixtures may be adjusted based on the specific application requirements. For example, the CO2 concentration may be varied between 1-10% for different cell culture protocols. The oxygen level may be reduced below 5% for more hypoxic (physioxic) conditions. Inert gases like argon or helium could also potentially be used in place of nitrogen for specialized applications. It is contemplated that the ability to precisely control gas composition through the multiple flow channels allows creation of complex gradients or layered gas environments.

In some embodiments, contemplated flow conditioners may produce a consistent non-turbulent or laminar air curtain that spans a vertical height in the range of 40-90 cm for standard laboratory applications. For smaller benchtop units, a vertical height of 30-45 cm may be suitable. Larger industrial-scale units may utilize a vertical height of 80-100 cm or greater. In further, more specified embodiments, the flow conditioning unit may have a vertical height of: 62.4 cm for a standard laboratory incubator, 40 cm for a compact benchtop incubator, 90 cm for a large-scale industrial incubator, 55 cm for a mid-size research incubator, and/or 70 cm for a high-capacity cell culture incubator. The vertical height may be adjustable within a range, such as 50-70 cm, to accommodate different applications. The height may also be customizable based on specific user requirements, with options ranging from 30 cm up to 120 cm or greater.

The system may be configured to achieve a range of air curtain angles, potentially including 1.0 degree, 5.0 degree, 7.5 degree, and 10.0 degree adjustments from the neutral position. The exact range of achievable angles may depend on various factors such as the flexibility and material properties of the apparatus components, servo motor specifications and torque capabilities, flow characteristics and velocities, and overall system geometry and constraints. Implementing precise angle control may allow for optimization of the air curtain barrier effect for different environmental conditions or chamber configurations. The ability to make fine adjustments to the flow angle may enhance the system's versatility and performance across various applications. For example, a slight downward angle may help counteract buoyancy effects from temperature differences, while an upward angle could enhance containment when the chamber door is opened.

In some embodiments, the numbers expressing quantities of ingredients, properties such as concentration, reaction conditions, and so forth, used to describe and claim certain embodiments of the invention are to be understood as being modified in some instances by the term “about.” As used herein, the terms "about" and "approximately", when referring to a specified, measurable value (such as a parameter, an amount, a temporal duration, and the like), is meant to encompass the specified value and variations of and from the specified value, such as variations of +/-10% or less, alternatively +/-5% or less, alternatively +/-1% or less, alternatively +/-0.1% or less of and from the specified value, insofar as such variations are appropriate to perform in the disclosed embodiments. Thus, the value to which the modifier "about" or "approximately" refers is itself also specifically disclosed. The recitation of ranges of values herein is merely intended to serve as a shorthand method of referring individually to each separate value falling within the range. Unless otherwise indicated herein, each individual value is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. As also used herein, and unless the context dictates otherwise, the term "coupled to" is intended to include both direct coupling (in which two elements that are coupled to each other contact each other) and indirect coupling (in which at least one additional element is located between the two elements). Therefore, the terms "coupled to" and "coupled with" are used synonymously.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification or claims refer to at least one of something selected from the group consisting of A, B, C …. and N, the text should be interpreted as requiring only one element from the group, not A plus N, or B plus N, etc.

What is claimed is:

1. An incubator, comprising:
a housing enclosing an internal container, wherein the housing and the internal container have an opening configured to allow access to an inside of the internal container;
a flow conditioner coupled to the housing and/or internal container and configured to form a composite air veil across the opening;
wherein the composite air veil has a thickness and a velocity gradient across the thickness; and
wherein a flow velocity difference between two adjacent sections within the thickness is below a shear force velocity difference.

2. The incubator of claim 1, wherein the flow conditioner includes a flow control element configured to receive an air flow and to induce a centrifugal or deflecting force in the air flow sufficient to produce a density gradient in the air flow to thereby produce a continuous velocity gradient across the thickness of the composite air veil.

3. The incubator of claim 1, wherein the flow conditioner includes a flow control element configured to receive an air flow and induce a pressure gradient in the air flow to thereby produce the velocity gradient across the thickness of the composite air veil.

4. The incubator of claim 1, wherein the flow conditioner is configured to form two composite air veils across the opening.

5. The incubator of claim 4, wherein a first of the two composite air veils has an ascending velocity gradient in a direction from the inside to an outside of the internal container, and wherein a second of the two composite air veils has a descending velocity gradient in the direction from the inside to the outside of the internal container.

6. The incubator of claim 1, wherein the thickness is between 1 cm and 10 cm, and/or wherein the shear force velocity difference is at least 0.4 m/s.

7. The incubator of claim 1, wherein the flow conditioner is configured to allow a portion of lower velocity air in the composite air veil to move into the internal container.

8. The incubator of claim 1, further comprising a recirculation conduit that is configured to recycle at least some of the air in the composite air veil.

9. The incubator of claim 1, wherein the recirculation conduit comprises tubing having a width or diameter that is less than a width of the opening of the internal container.

10. The incubator of claim 1, wherein the incubator is a controlled atmosphere incubator.

11. The incubator of claim 1, wherein the composite air veil has a composition other than ambient air.

12. The incubator of claim 1, further comprising a control system that is configured to adjust a flow rate of air in the composite air veil.

13. The incubator of claim 12, wherein the control system is further configured to maintain an atmospheric parameter in the internal container.

14. A method of maintaining an atmospheric condition within an incubator having a housing that encloses an internal container, wherein the housing and the internal container have a door covering an opening that allows access to an inside of the internal container, the method comprising:
operating a flow conditioner when the door is in an open position, wherein the flow conditioner forms a composite air veil moving across the opening;
wherein the composite air veil has a thickness and a velocity gradient across the thickness; and
wherein a flow velocity difference between two adjacent sections within the thickness is below a shear force velocity difference.

15. The method of claim 14, wherein the composite air veil has a symmetrical velocity gradient.

16. The method of claim 14, further comprising operating a second flow conditioner that forms a second composite air veil moving substantially parallel to the composite air veil moving across the opening.

17. The method of claim 14, wherein the flow conditioner includes a flow control element that receives an air flow and induces a centrifugal or deflecting force in the air flow sufficient to produce a density gradient in the air flow to thereby produce a continuous velocity gradient across the thickness of the composite air veil.

18. The method of claim 14, wherein the flow conditioner includes a flow control element that receives an air flow and induces a pressure gradient in the air flow to thereby produce the velocity gradient across the thickness of the composite air veil.

19. The method of claim 14, further comprising a step of using a control system to control air flow in the composite air veil, and/or further comprising a step of using the control system to control composition of air in the composite air veil.

20. The method of claim 14, wherein a flow velocity in a fastest section of the composite air veil is above 2 m/s.
